(19) United States
(12) Patent Application Publication
(10) Pub. No.: US 2023/0145671 A1
(43) Pub. Date: May 11, 2023

(54) Wearable electronic device

(71) Applicant: Suunto Oy, Vantaa (FI)

(72) Inventors: Britta Stockinger, Vantaa (FI); Jere Lallo, Vantaa (FI); Atte Kuparinen, Vantaa (FI); Jouni Junes, Vantaa (FI); Erik Lindman, Vantaa (FI)

(21) Appl. No.: 17/967,928

(22) Filed: Oct. 18, 2022

(30) Foreign Application Priority Data
Nov. 5, 2021 (FI) 20216139

(51) Int. Cl. A44C 5/00; A44C 5/14

(52) U.S. Cl. CPC A44C 5/0053; A44C 5/14

(57) ABSTRACT

It is herein disclosed a novel strap for a wearable electronic device and a wearable electronic device with such a strap. The strap features a first layer of microfiber and a second layer of natural or artificial leather as an outer layer. A layer of elastic adhesive attaches the first layer to the second layer.

### FIELD

The present disclosure relates to apparatuses affixing a wearable electronic device to the user's anatomy. In particular the disclosure relates to straps for the purpose of providing a snug fit for an electronic wearable device comprising an optical heart rate sensor in order to improve heart rate measurements.

### BACKGROUND

The fit of a strap for a wearable electronic device is important not only for providing a comfortable using experience but also for ensuring reliable operation of the device. Several attempts have been made to improve the fit of a strap by optimizing its stretching properties, as discussed in US 20160255921 A1 and US20170065038A1.

A challenge of designing contemporary smart wearable devices is the need to use a particular material in the strap. Leather, in particular, is appreciated by consumers for its durability but also for aesthetic reasons. However, both natural and artificial leather grades have turned out be far from optimal as the base material of a strap for a wearable electronic device because leather has limited compliance properties for ensuring a snug fit.

Accordingly there remains a need to develop a leather strap for a wearable electronic device that would provide for a good fit required by sensors of the electronic device.

### SUMMARY

The invention is defined by the features of the independent claims. Some specific embodiments are defined in the dependent claims.

According to a first aspect of the present disclosure, there is provided a wearable electronic device with a strap. The strap features a first layer of microfiber and a second layer of natural or artificial leather as an outer layer. A layer of elastic adhesive attaches the first layer to the second layer. The first layer is a structural part of the strap, which structural part dominantly defines the mechanical properties of the strap, whereas the second layer is a façade complying to the first layer. The thickness of the layer of elastic adhesive is smaller than the thickness of the first layer and second layer. The wearable electronic device comprises an optic heart rate sensor housed in the enclosure.

Certain embodiments of the invention may include one or more features from the following list:
the microfiber base material is an ultra-microfiber fabric
the microfiber base material is Ultrasuede®;
the blank of the first layer consists of a single piece ultra-microfiber fabric;
the first layer is set up as an inner layer opposing the outer layer;
the second layer is thinner than the first layer;
the elastic properties of the first layer and the second layer are substantially matched with each other;
the second layer is more flexible than the first layer;
the strip is elongated along a longitudinal dimension and has a width in a transversal dimension;
the strip exhibits greater elasticity in the longitudinal dimension than in the transversal dimension;
the difference in elasticity between the longitudinal dimension and the transversal dimension is 20% or more;
the difference in elasticity between the longitudinal dimension and the transversal dimension is 50% or more;
the difference in elasticity between the longitudinal dimension and the transversal dimension is 100% or more;
the difference in elasticity between the longitudinal dimension and the transversal dimension is 200% or more;
the difference in elasticity, particularly in elastic modulus and/or along the longitudinal dimension, between the first layer and second layer is at most 20%;
the difference in elasticity, particularly in elastic modulus and/or along the longitudinal dimension, between the first layer and the layer of elastic adhesive is at most 20%;
the adhesive layer is more flexible than the first layer;
the thickness of the first layer in the range of 1 to 2 mm;
the thickness of second layer is less than 1 mm;
the strip comprises only the first layer, the second layer and the layer of elastic adhesive across the whole strip;
the strip comprises only the first layer, the second layer and the layer of elastic adhesive across the whole strip in the longitudinal and transversal dimension, whereby the strip may comprise further elements in the thickness dimension;
the strip comprises an attachment end with a loop section for forming a space for a fixture to the wearable electronic device;
the strip comprises a closing end with a loop section for forming a space for a fixture to a buckle;
the strip comprises both such an attachment end (and such a closing end;
the second layer covers the loop section;
the strip comprises a seam, which terminates the loop section and forms a transition between the second layer and the first layer;
the first layer is exposed throughout most of the inside of the strap;
the strap comprises a first part, which has an attachment end and a closing end;
the strap comprises a second part, which has an attachment end and a closing end;
the attachment end of the first part is configured to attach to the wearable electronic device at a first attachment point on the wearable electronic device;
the closing end of the first part is configured to be attached to the closing end of the second part;
the attachment end of the second part is configured to attach to the wearable electronic device at a second attachment point on the wearable electronic device;
the strap comprises a fixture inserted into the loop section of the strip;
the strap comprises stitching in the vicinity of the closing end of the first part or the loop section of the first part or second part or in any combination thereof;
the first part comprises a buckle and a cooperative tongue at the closing end;
the second part comprises a corresponding tip, which is configured to be inserted through the buckle, and a plurality of holes for receiving the tongue;
the wearable electronic device comprises an enclosure;
the enclosure comprises a first attachment point provided at one end of the enclosure;
the first attachment point is configured to receive the loop section of the first part of the strap through a fixture;
the enclosure comprises a second attachment point provided at another end of the enclosure;
the second attachment point is configured to receive the loop section of the second part of the strap through a fixture;
the attachment points are both configured to receive a spring bar,
the second layer has at most 30%, at most 20% or less than 20% impact on the mechanical properties of the strip,
majority of the inner layer is exposed,
only the minority of the surface area of the inner layer is covered.

Considerable benefits are gained with aid of the present invention.

By using leather as the façade outer layer of a laminated strap, the benefits of leather may be had while pursuing technical properties that are beneficial for smart devices requiring reliable skin contact. On the other hand, by using microfiber material as the raw material of the inner layer of strap, elasticity is gained in one dimension and lost in another. This enables orientation of the strap fabric such that, once closed into a loop, the elasticity of the strap ensures a reliable fit of the wearable electronic device on the user. The relatively soft microfiber material of the strap, in turn, facilitates compliance with minor contours on the users anatomy, such as protruding bones, etc. By having relatively little elasticity in the transversal dimension, the strap maintains a good deformation resistance. Such benefits are particularly useful in applications in which the wearable electronic device features an optical heart rate sensor, the accuracy of which is greatly dependent on a uniform contact with the user's skin. Optical heart rate or pulse measurement is performed with a sensor arrangement with light emitters and light sensors placed at the watch case bottom. The measurement is disturbed by ambient light reaching the sensor or sensors from the sides if the watch does not stay in good contact with the skin. Also the oscillations or vibrations of the wrist tissue at the sensors from when the wrist is moving, for example when running, causes signal noise thus making the measurement more difficult. The problems with the reliability of the measurement are worsened if the watch is relatively heavy and loosely fit. On the other hand, an overly tight watch strap would be too uncomfortable. It is therefore desirable to have a snug fit with a comfortable feel in order to make a desirable product with good quality. In other words, the novel strap construction has the potential in improving the accuracy of heart rate signal acquired with optical heart rate sensors enclosed in a wearable electronic device.

These benefits could not be gained by constructing the strap entirely of leather.

Additionally, the inner layer, which is in permanent contact with the skin, may be cleaned with effective substances that could not be applied to leather.

### BRIEF DESCRIPTION OF THE DRAWINGS

In the following certain exemplary embodiments are described in greater detail with reference to the accompanying drawings, in which:

FIG. 1 illustrates a top elevation view of a strap in accordance with at least some embodiments of the present invention connected to an electronic wearable device shown in dashed lines in a flat configuration;

FIG. 2A illustrates a top elevation view of the short piece of the strap of FIG. 1;

FIG. 2B illustrates a side elevation view of the short piece of the strap of FIG. 1;

FIG. 2C illustrates a bottom elevation view of the short piece of the strap of FIG. 1,

FIG. 3A illustrates a top elevation view of the long piece of the strap of FIG. 1;

FIG. 3B illustrates a side elevation view of the long piece of the strap of FIG. 1;

FIG. 3C illustrates a bottom elevation view of the long piece of the strap of FIG. 1, and

FIG. 4 illustrates a side elevation view of the strap and electronic wearable device of FIG. 1 in a loop configuration.

### EMBODIMENTS

In the following numerous embodiments are described with a strap featuring a strip made from at least two materials layered on each other as a structural layer and as a façade layer. The façade layer is intended as a decorative cover layer, which is not supposed to provide structural load carrying properties to the strap. Accordingly, the façade layer is preferably more flexible than the structural layer. The structural layer and the façade layer are attached to each other with an adhesive layer, wherein the adhesive and façade layer are preferably set to withstand, i.e. not break at, the maximum designed flex or deformation of the strap. Throughout this description the structural layer is referred to as an inner or first layer and the decorative cover layer is referred to as a façade or second layer.

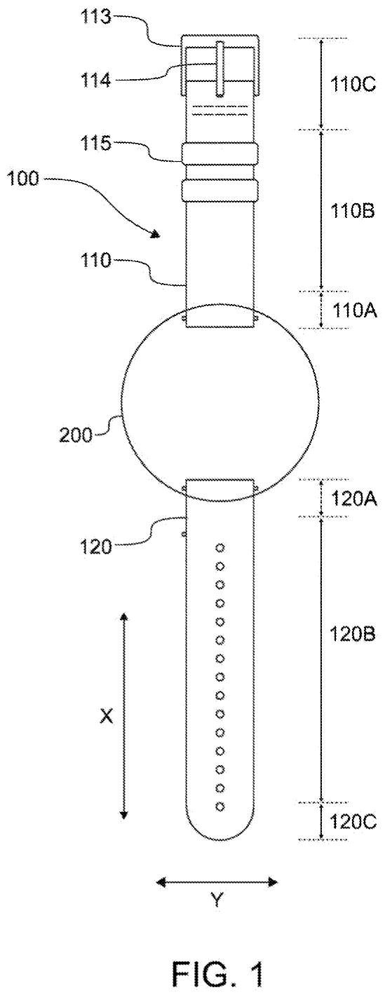

FIG. 1 illustrates an exemplary strap 100 attached to a wearable electronic device 200 shown in dashed line according to an embodiment. The strap and wearable electronic device 200 is presented in a flat configuration with the strap extending straight from the wearable electronic device. The embodiment is described in the form of a wrist band for affixing a smart watch to the wrist of a user. The principles herein described and defined in the appended claims are applicable to other terminal devices, such as heart rate monitors or motion sensors, affixed to different parts of human anatomy, such as the chest, ankle, or upper arm. Furthermore the illustrated examples present a buckle mechanism for attaching the strap pieces 110, 120 to each other but the buckle mechanism could be replaced with a different closing mechanism, such as a butterfly clasp. The embodiments share the central purposeful concept of the strap 100 or strap pieces 110, 120 including a leather façade layer and a microfiber inner layer, particularly ultra-microfiber, such as Ultrasuede®.

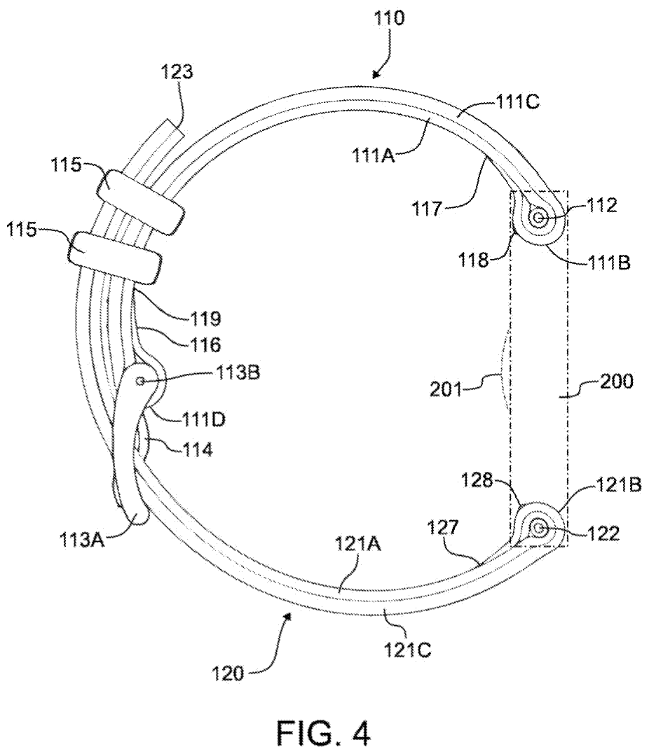

Let us first turn to the strap 100, which is shown in a planar spread-out configuration in FIG. 1 and in a loop configuration in FIG. 4. The strap 100 is made up by two parts, namely a first part 110 and a second part 120. Both parts 110, 120 feature a strip 111, 121 equipped with ancillary elements, such as fixtures. The strip 111 is elongated along a longitudinal dimension X, has a width in a first transversal dimension Y and a thickness in another transversal dimension. The wearable electronic device 200, such as a smart watch, is provided and affixed to between the parts 110, 120. The first part 110 acts as the female part of a two-part strap and comprises a buckle 113 with a tongue 114 for engaging a respective opening in the second part 120. In the present field, such strap parts are referred to as “short pieces”. As indicated above, the buckle 113 could be replaced with a different mechanism, such as a simple loop for a Velcro attachment (not illustrated in the FIGURES), a clasp part (not illustrated in the FIGURES) or a positively engaging quick coupler (not illustrated in the FIGURES). The first part 110 has three sections, namely an attachment end 110A at one end for attachment to the wearable electronic device 200, a closing end 110C at the other end for accommodating the attachment to the second strap part 120, and an intermediate section 110B there between. The first part 110 may also include conventional loops 115 for keeping excess sections of the second strap part 120 in a loop configuration of the strap (FIG. 4).

The second part 120 acts as the male part of a two-part strap and comprises a tip 123 for entering through the buckle 113 and a sequence of holes 124 for receiving the a tongue 114. In the present field, such strap parts are referred to as “long pieces”. As indicated above, the buckle mechanism could be replaced with an alternative, whereby the second part 120 would include a Velcro attachment piece, a magnet, a clasp part, a quick coupler, etc. The second part 120 has three sections, namely an attachment end 120A at one end for attachment to the wearable electronic device 200, a closing end 120C at the other end for accommodating the attachment to the first strap part 110, and an intermediate section 120B there between.

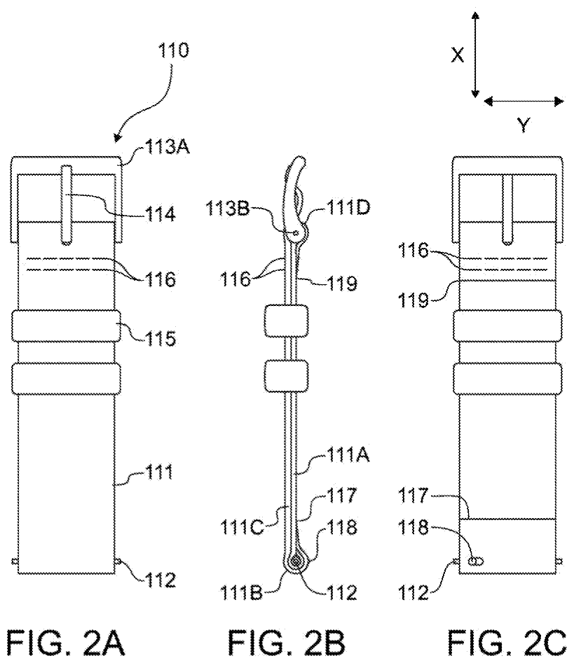

FIGS. 2A, 2B, and 2C show details of the first part 110. FIG. 2B shows the first part 110A from the side revealing the double-layer construction of the strap. The first layer 111A faces the user and is in constant skin contact with the user. The first layer 111A extends between the loop ends 111B and 111D and therefore covers most of the first part 110 along the longitudinal dimension. The first layer 111A is exposed on the inside of the strap 100.

The first layer 111A of the strip 111 is made from microfiber. In other words, the first layer 111A comprises microfiber base material. The first layer 111A, when processed into a part of the strap part, may include further materials, such as glue or texture yarn or silicone pads attached to the inner surface, but the base of the strip is formed by a microfiber cloth. The thickness of the first layer 111A may be at most 3 mm, such as in the range of 1 to 2 mm. The microfiber material is preferably ultra-microfiber, such as Ultrasuede®. The microfiber material may comprise polyester or polyamide or other suitable fiber material, such as carbon fiber, and polyurethane or other suitable flexible polymer as a binder, such as silicone.

The first layer 111A extends along the entire longitudinal dimension X of the strip 111. The first layer 111A may optionally, as shown, extend through the loop sections 111B, 111D. Alternatively, the first layer 111A may terminate before the loop section 111B, 111D or either one of them. FIG. 2B shows how the first layer 111A loops around a spring bar 112 and pin 113B of the buckle 113A. The spring bar 112 acts as an exemplary fixture to the electronic device 200. Other fixture alternatives include snap connectors, sliding blocks, etc. On the inside, the first layer 111A is attached to itself. The end of the first layer 111A is preferably chamfered as thin as possible and along a suitable length to provide for a large surface area for the attachment agent. The same applies to both ends of the strip 111. The loop section 111D at the closing end 110C also has a similar looped ending as the loop section 111B at the attachment end 110A. The embodiment shown in FIG. 2B, in which the first layer 111A covers the loop section 111B may be preferred for the sake of the relatively wear-resistant and slippery microfiber is in contact with the hardware contained in the loop sections 111B, 111D.

According to a particular embodiment the microfiber comprises 65 to 80 weight-% of polyester ultra-microfiber which is non-woven with 35 to 20 weight-% of non-fibrous polyurethane binder.

The first layer 111A extends along the longitudinal dimension X to form an inner layer of the strip. The first layer 111A is oriented such that it exhibits greater elasticity in the longitudinal dimension X than in the transversal dimension Y. The difference in the elastic modulus between the longitudinal dimension X and the transversal dimension Y may be 20% or more, preferably 50% or more, 100% or more, 200% or more, or by more than one or two order of magnitude.

The strip 111 features a second layer 111C made of natural or artificial leather forming the façade of the strap 100. Suitable types of leather include cow leather skived into a relatively thin layer. The thickness of the second layer 111C is quite small so that it has a minimal effect on the elasticity of the strip 111. Accordingly, the thickness of the second layer 111C may be less than 1 mm, such in the range of 0.2 to 0.9 mm, particularly between 0.5 and 0.8 mm.

The second layer 111C forms the façade of the strip 111 and, thus, faces away from the user, whereby it is not subjected to skin oils. To fully cover the visible part of the strip 111, the second layer 111C extends along the entire longitudinal dimension X of the strip 111 but also through the loop sections 111B, 111D. FIG. 2B shows how the second layer 111C loops around the spring bar 112 and pin 113B of the buckle 113A. On the inside, the second layer 111C is attached to the first layer 111A through a seam 117, which terminates the loop section 111B but also transitions the second layer 111C into the first layer 111A and outer layer 111C. To enable a smooth transition, the end of the second layer 111C is preferably chamfered as thin as possible and along a suitable length to provide for a large surface area for the attachment agent. The same applies to both ends of the strip 111. The loop section 111D at the closing end 110C also has a similar seam 119 as the loop section 111B at the attachment end 110A.

In other words, the inner layer 111A is intended to face the user and be in contact with the user's skin. For this purpose, majority of the inner layer 111A is exposed. In other words, only the minority of the surface area of the inner layer 111A may, according to one embodiment, be covered.

The layers 111A, 111C are attached to each other by gluing for example with a thermosetting adhesive there between. Accordingly, there is a layer of elastic adhesive or heat activated bonding layer between and the layers 111A, 111C fixing the layers 111A, 111C together. The layer of elastic adhesive is very thin, at least thinner than first and second layer 111A, 111C. It is preferred that the layer of elastic adhesive is so thin that it cannot be observed with the naked eye with unimpeded vision at a 20 cm distance in broad daylight. The adhesive may penetrate in part or entirely into the layers 111A, 111C. The adhesive selected is preferably strong and compliant to allow the strip 111 to stretch in the longitudinal dimension. Suitable adhesives include thermosetting adhesives, such as a thermoplastic polyurethane film that is melt to activate the bonding properties. Other examples include adhesives that are applied in liquid form and can be cured by drying or heating. It is preferable that the selected adhesive is at least as elastic as the microfiber of the first layer 111A, 121A so as to prevent delamination, when exposed to stretch. It is at least preferable that the adhesive is not a limiting factor in the stretching ability of the strap 100. During manufacturing a blank of microfiber and leather may be laminated into two layers with elastic glue or heat activated bonding layer and then cut to the final shape or the strip is first cut from blanks of microfiber and leather, which are then processed into the shape shown in the FIGURES.

As previously mentioned, the structural layer and the façade layer are attached to each other with an adhesive layer, wherein the adhesive and façade layer are preferably set to withstand, i.e. not break at, the maximum designed flex or deformation of the strap. This can be tested so that one measures the flex of the strap for example in a 200 kN static strap test. Accordingly, the façade layer may be tested to ensure that it is able endure the measured stretch of the strap. The exemplary 200 kN test force can be replaced with any test that measures the flex of the strap in the foreseen use purpose. In other words, the strap is designed to exhibit an inherent flex. Only several flex cycles ensure that the façade layer does not suffer damage or become delaminated.

The adhesive attachment may be reinforced with stitching, particularly at the vicinity of either or both of the loop sections 111B, 111D. In the illustrated example only the loop section 111D at the closing end 110C is provided with a double-line stitching that penetrates both layers 111A, 111C.

The first layer 111A, 121A is a structural part of the strap 100 that dominantly defines the mechanical properties of the strap 100. The second layer 111C, 121C, on the other hand, is merely a façade that complies to the properties of the strap largely set set by first layer 111A, 121A. It may be seen that the second layer 111C, 121C has at most 20% impact on the mechanical properties of the strap 100. For example, if a property of first layer 111A, 121A, such as stretch properties, flexibility, or tensile strength, is measured both in isolation of the second layer 111C, 121C and with the second layer 111C, 121C, the difference of the measured values without the second layer compared to measured values with the second layer may be at most 30%, preferably at most 20%, most preferably less than 20%.

The first part houses fixture 112, such as a spring bar, within the strip 111 enclosed by the loop section 111B. The spring bar 112 is used for attachment to the wearable electronic device 200. The spring bar 112 may be operated with a release mechanism 118 for toggling the movable pin of the spring bar 112 between a deployed and retracted state. The release mechanism 118 may be accessible through a respective opening provided to the strip 111. The buckle 113 comprises a comparable bar (not shown in the FIGURES), around which the loop section of the strip 111 is wound. The preferably beveled ends of the strip 111 meet at a seam 117, 119 which is closed by gluing. According to another embodiment, the ends of the strip 111 at the seam 117, 119 is closed by a thermoset adhesive. According to another embodiment, the ends of the strip 111 at the seam 117, 119 is closed by contact glue. According to another embodiment, the ends of the strip 111 at the seam 117, 119 is closed by welding. According to another embodiment, the ends of the strip 111 at the seam 117, 119 is closed by sewing.

According to another embodiment, in which the second layer 111C is made of artificial leather, such as one comprising PVC and/or PU, the ends of the strip 111 at the seam 117, 119 is closed by melting the layers 111A, 111C together by applying heat. In particular, the artificial leather may comprise a polyester substrate with a thermoplastic polyurethane top layer that provides soft feel and texture that resembles that of natural leather. Indeed the artificial leather is selected from a synthetic material that mimics the properties and surface texture of natural leather. The synthetic material may be a soft elastomer material, such as silicone. The artificial leather may alternatively comprise natural base material, such as coated banana leaves or coated fiber extracted from banana leaves.

The thickness of the second layer 111C, 121C is preferably set to match the stretching properties of the first layer 111A, 121A. As microfiber is inherently more elastic than natural or synthetic leather, the thickness of second layer 111C, 121C is relatively small compared to the first layer 111A, 121A. Absolute matching of stretching properties between the layers is not required but it is preferable to set the difference in elasticity between the first layer 111A, 121A and second layer 111C, 121C at most 20%. In other words, the difference in elastic modulus along the longitudinal dimension X is preferably at most 20% between the layers 111A, 121A; 111C, 121C.

As the second layer 111C, 121C and the adhesive layer between the first and second layer are noticeably thinner than the first layer 111A, 121A, it is foreseen that the second layer 111C, 121C or the adhesive layer or both the second layer 111C, 121C and the adhesive layer is or are more flexible than the first layer 111A, 121A. Accordingly, the flexibility of the first layer 111A, 121A is decisive for the flexibility of the strap 100. In other words, the properties of the first layer 111A, 121A dominate the properties of the strap 100A.

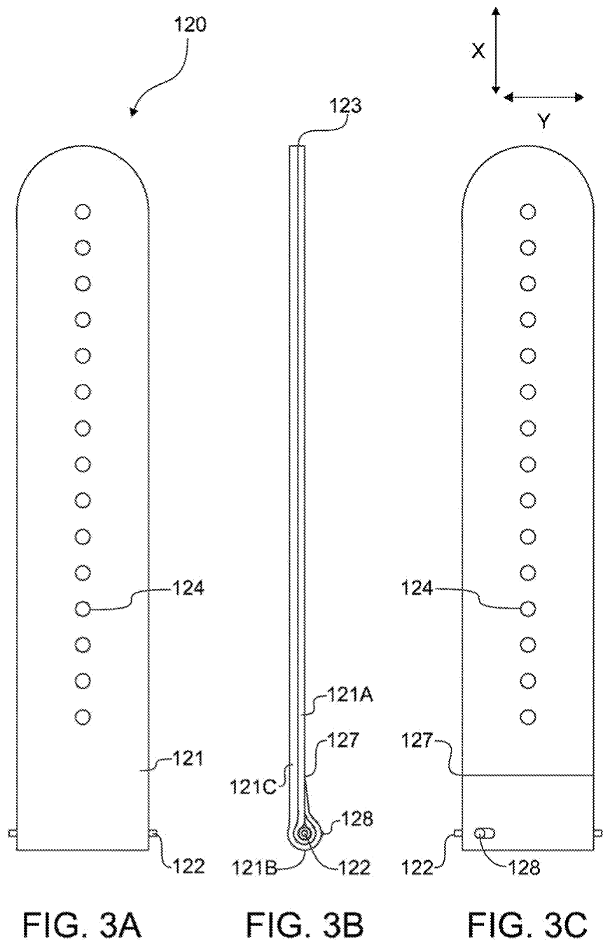

FIGS. 3A, 3B, and 3C show details of the second part 120. FIG. 2B shows the first part 110A from the side revealing the construction of the strap which is largely similar to that of the first part 110. The first layer 121A extends along the longitudinal dimension X to form an inner layer such oriented that it exhibits greater elasticity in the longitudinal dimension X than in the transversal dimension Y. The difference in the elastic modulus between the longitudinal dimension X and the transversal dimension Y may be 20% or more, 50% or more, 100% or more, 200% or more or by one or two order of magnitude or more. The strip 121 features a parallel outer layer 121C made of natural or artificial leather thin enough and similarly oriented so as to maintain the orientation of the stretching ability along the longitudinal dimension X. The strip 121 transitions between the inner layer 121A and outer layer 121C at a loop section 121B at the attachment end 120A of the second part 120 for housing a spring bar 122. The ends of the strip 121 meet at the tip 123 at the closing end 120C. In other words the strip 121 is wound into two superposed layers 121A, 121C at each point in the strip 111. The layers 121A, 112C are attached to each other by gluing for example with a thermosetting adhesive there between. The attachment may be additionally secured at the vicinity of the loop section 121B and/or at the tip 123 with stitching 127, thermoset gluing, welding, or sewing. The closing end 120C may also include stitching for preventing the ends of the strip 121 from becoming detached from each other. A series of subsequently positioned holes 124 have been provided through the strip 121 along the longitudinal dimension for receiving the tongue 114 of the buckle 113 of the first part 110A.

The exemplary wearable electronic device 200 takes the form a smart watch. The enclosure of the wearable electronic device 200 includes two attachment points at opposing ends of the enclosure; one for each spring bar 112, 122 of the strap parts 110, 120. Naturally, the spring bar attachment could be replaced with other foreseeable attachment mechanisms, such as affixer-secured or clenched bars, sliding coupler parts in a corresponding attachment groove on the enclosure, magnets, etc.

The enclosure of the wearable electronic device 200 preferably also includes an optical heart rate sensor 201, whereby the benefits of the novel strap may be utilized for the purpose of ensuring a reliable fit between the wrist of the user and the sensor optics. Optical heart rate or pulse measurement is performed with a sensor arrangement with light emitters and light sensors placed at the watch case bottom. The measurement is disturbed by ambient light reaching the sensor or sensors from the sides if the watch does not stay in good contact with the skin. Also the oscillations or vibrations of the wrist tissue at the sensors from when the wrist is moving, for example when running, causes signal noise thus making the measurement more difficult. The problems with the reliability of the measurement are worsened if the watch is relatively heavy and loosely fit. On the other hand, an overly tight watch strap would be too uncomfortable. It is therefore desirable to have a snug fit with a comfortable feel in order to make a desirable product with good quality.

The use of the strap 100 is straight-forward. The wearable electronic device 200 is placed on the desired anatomic location of the user, such as the wrist. The strap parts 110, 120 are coupled to each other by inserting the tip 123 through the buckle 114, by pulling a desired amount of tension into the strap 100 and securing the strap into a loop around the anatomic location by inserting the tongue 114 into a corresponding hole 124 of the second strap part 120. Once closed into a loop, the elasticity of the strap 100 along the longitudinal dimension X ensures a reliable fit of the wearable electronic device 200 on the user. The microfiber material of the strap 100, in turn, facilitates compliance with minor contours on the users anatomy, such as protruding bones, etc. By having relatively little elasticity in the transversal dimension, the strap maintains a good deformation resistance. The relatively small stretch in the transversal dimension Y, i.e. along the width of the strap, facilitates sturdy attachment to the hardware of the device, e.g. to the spring bar and buckle. If the strap would be relatively compliant in the transversal dimension Y, the excess elasticity could compromise attachment to the wearable electronic device. The relative resistance to elastic deformation in the transversal dimension Y minimizes fatigue in the adhesive layer between the strap layers 111A, 111C; 121A, 121C.

The strap 100 may be further enhanced by including a reflective yarn pattern, an embedded auxiliary battery, etc. The base material of the strap may be treated with a anti-bacterial supplement for making the strap more suitable for a sporting device application.

In the examples described with reference to FIGS. 1 to 4 a construction is proposed with the first layer 111A, 121A, the second layer 111C, 121C and the layer of elastic adhesive extending across the whole strap 100 in the longitudinal and transversal dimensions X, Y. It is, however, possible to include further components to the strap, such as a chip, particularly an RFID or NFC chip, or an antenna between the layers. While such a component may be placed at some location of the strap, it is foreseen that said component is only locally present and does not extend across the whole strap similarly to the structural layers.

According to an alternative embodiment, the strip may comprise a third layer positioned on the inside of the strip, i.e. attached against the inner layer as an inner façade layer for skin contact. While this embodiment may not achieve the hygiene benefits of microfiber, it may provide a familiar leather “feel” preferred by some users. It is nevertheless preferred that the additional inner façade layer shares its properties with the outer façade layer so as to not compromise the stretch properties predominantly defined by the inner microfiber layer sandwiched between the façade layers. By applying an inner and outer façade leather layer the stretch properties of the strip may be affected approximately 20 to 30 percent. According to the tri-layer embodiment, the inner façade layer may extend across the strip between the ends thereof or as a patch adhered to and covering the inner microfiber layer, whereby either or both ends of the strip would be covered by the outer façade layer extending over the end.

Conversely, according to a particular embodiment, the layered structure of the strip consists of only three layers, i.e. the first layer 111A, 121A facing the user, the second layer 111C, 121C as an outer layer, and the layer of elastic adhesive there between. Naturally, the strap may include hardware, such as a buckle, and/or reinforcing stitching, which are not seen as components of the layered structure.

It is also foreseen to attach the layers together by welding along transversal seams, if the artificial leather is constructed of raw material, which enables welding. Such an embodiment could be constructed without the elastic adhesive layer.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### REFERENCE SIGNS LIST

| No. | Feature |
|---|---|
| 100 | strap |
| 110 | first part, “short piece” |
| 110A | attachment end |
| 110B | intermediate section |
| 110C | closing end |
| 111 | strip |
| 111A | inner layer |
| 111B | loop section |
| 111C | outer layer |
| 111D | loop section |
| 112 | spring bar |
| 113A | buckle |
| 113B | pin |
| 114 | tongue |
| 115 | loop |
| 116 | stitching for buckle |
| 117 | seam |
| 118 | release mechanism |
| 119 | seam |
| 120 | second part, “long piece” |
| 120A | attachment end |
| 120B | intermediate section |
| 120C | closing end |
| 121 | strip |
| 121A | inner layer |
| 121B | loop section |
| 121C | outer layer |
| 122 | spring bar |
| 123 | tip |
| 124 | hole |
| 127 | seam |
| 128 | release mechanism |
| 200 | wearable electronic device |
| 201 | optical heart rate sensor |
| X | longitudinal dimension |
| Y | transversal dimension |

### CITATION LIST

### Patent Literature

US 20160255921 A1
US 20170065038 A1

1. A wearable electronic device comprising a strap, wherein the strap comprises a strip with:
a first layer,
a second layer as an outer layer comprising a natural or artificial leather base material, which second layer is superposed on the first layer, and
a layer of elastic adhesive attaching the first layer to the second layer,
wherein:
the first layer comprises microfiber base material,
the first layer is a structural part of the strap dominantly defining mechanical properties of the strap whereas the second layer is a façade complying to the first layer,
the thickness of the layer of elastic adhesive is smaller than the thickness of the first layer and second layer, and
the wearable electronic device comprises an enclosure and an optic heart rate sensor housed in the enclosure.

2. The wearable electronic device according to claim 1, wherein the enclosure of the wearable electronic device comprises:
a first attachment point provided at one end of the enclosure, which first attachment point is configured to receive the loop section of the first part of the strap through a fixture, and
a second attachment point provided at another end of the enclosure, which second attachment point is configured to receive the loop section of the second part of the strap through a fixture.

3. The wearable electronic device according to claim 1, wherein the microfiber base material is an ultra-microfiber fabric, such as Ultrasuede®.

4. The wearable electronic device according to claim 1, wherein a blank of the first layer consists of a single piece ultra-microfiber fabric.

5. The wearable electronic device according to claim 1, wherein the first layer is set up as an inner layer opposing the outer layer.

6. The wearable electronic device according to claim 1, wherein the second layer is thinner than the first layer.

7. The wearable electronic device according to claim 1, wherein:
the elastic properties of the first layer and the second layer are substantially matched with each other, or wherein
the second layer or the adhesive layer or both the second layer and the adhesive layer is or are more flexible than the first layer.

8. The wearable electronic device according to claim 1, wherein:
the strip is elongated along a longitudinal dimension and has a width in a transversal dimension, and
the strip exhibits greater elasticity in the longitudinal dimension than in the transversal dimension.

9. The wearable electronic device according to claim 8, wherein the difference in elasticity between the longitudinal dimension and the transversal dimension is:
20% or more,
50% or more,
100% or more, or
200% or more.

10. The wearable electronic device according to claim 8, wherein the difference in elasticity, particularly in elastic modulus and/or along the longitudinal dimension, between the first layer and second layer is at most 20%.

11. The wearable electronic device according to claim 8, wherein the difference in elasticity between the first layer and the layer of elastic adhesive is at most 20%.

12. The wearable electronic device according to claim 1, wherein the thickness of the first layer in the range of 1 to 2 mm.

13. The wearable electronic device according to claim 12, wherein the thickness of second layer is less than 1 mm.

14. The wearable electronic device according to claim 1, wherein the strip comprises only the first layer, the second layer and the layer of elastic adhesive across the whole strip.

15. The wearable electronic device according to claim 1, wherein the strip comprises:
an attachment end with a loop section for forming a space for a fixture to the wearable electronic device, or
a closing end with a loop section for forming a space for a fixture to a buckle, or
both such an attachment end and such a closing end.

16. The wearable electronic device according to claim 15, wherein:
the second layer covers the loop section, and
the strip comprises a seam terminating the loop section and forming a transition between the second layer and the first layer.

17. The wearable electronic device according to claim 1, wherein the first layer is exposed throughout most of the inside of the strap.

18. The wearable electronic device according to claim 15, wherein the strap comprises a fixture inserted into the loop section of the strip.

19. The wearable electronic device according to claim 8, wherein the difference in elastic modulus along the longitudinal dimension, between the first layer and the layer of elastic adhesive is at most 20%.
